(19) United States
(12) Patent Application Publication
(10) Pub. No.: US 2020/0335216 A1
(43) Pub. Date: Oct. 22, 2020

(54) MEDICAL INFORMATION PROCESSING APPARATUS, MEDICAL INFORMATION PROCESSING SYSTEM, AND MEDICAL INFORMATION PROCESSING METHOD

(71) Applicant: CANON MEDICAL SYSTEMS CORPORATION, Otawara-shi (JP)

(72) Inventors: Aina IKEZAKI, Utsunomiya (JP); Tetsuo SHIMADA, Hachioji (JP); Shigeharu OHYU, Yaita (JP)

(73) Assignee: CANON MEDICAL SYSTEMS CORPORATION, Otawara-shi (JP)

(21) Appl. No.: 16/847,855

(22) Filed: Apr. 14, 2020

(30) Foreign Application Priority Data
Apr. 18, 2019 (JP) 2019-079478

(51) Int. Cl. G16H 50/20; G16H 70/20; G16H 40/20; G16H 30/40; G16H 40/40; G06Q 10/06; G06T 7/00

(52) U.S. Cl. CPC G16H 50/20; G16H 70/20; G16H 40/20; G06T 7/0012; G16H 40/40; G06Q 10/06316; G16H 30/40

(57) ABSTRACT

A medical information processing apparatus according to an embodiment includes processing circuitry. The processing circuitry is configured to receive, with respect to each of a plurality of examinations for diagnosing a disease suspected in a subject, first information indicating whether or not it is necessary to obtain confirmation from a doctor before the examination is performed and second information indicating whether or not it is necessary to obtain consent from the subject or an appropriate representative before the examination is performed. The processing circuitry is configured to set each of the plurality of examinations with the first information and the second information as additional information. The processing circuitry is configured to control an examination order including the plurality of examinations on the basis of the additional information.

### CROSS-REFERENCE TO RELATED APPLICATIONS

This application is based upon and claims the benefit of priority from Japanese Patent Application No. 2019-079478, filed on Apr. 18, 2019; the entire contents of which are incorporated herein by reference.

### FIELD

Embodiments described herein relate generally to a medical information processing apparatus, a medical information processing system, and a medical information processing method.

### BACKGROUND

In medical facilities such as hospitals, medical doctors (hereinafter, “doctors”) issue examination orders to perform medical examinations (hereinafter, “examinations”) on examined subjects (hereinafter, “subjects”), so that staff who is in charge of examinations (hereinafter “examination staff”) receives the examination orders and performs the examinations on the subjects. In these situations, each doctor reviews the examination results and issues another examination order to perform a next examination on the subject. However, when the doctors issue the next examination order after the examination results become available, because it takes time for the doctor to issue the next examination order after the issuance of the first examination order, burdens are imposed on the doctors, the examination staff, the subjects, and the like.

### BRIEF DESCRIPTION OF THE DRAWINGS

FIG. 1 is a diagram illustrating an exemplary configuration of a medical information processing system including a medical information processing apparatus according to the present embodiments;

FIG. 2 is a flowchart illustrating a processing procedure performed by a medical information processing system according to a first embodiment;

FIG. 3 is a drawing illustrating an examination order input screen according to the first embodiment;

FIG. 4 is another drawing illustrating the examination order input screen according to the first embodiment;

FIG. 5 is yet another drawing illustrating the examination order input screen according to the first embodiment;

FIG. 6 is yet another drawing illustrating the examination order input screen according to the first embodiment;

FIG. 7 is a flowchart illustrating an example of processes based on additional information;

FIG. 8 is a flowchart illustrating another example of the processes based on the additional information;

FIG. 9 is a flowchart illustrating a processing procedure performed by a medical information processing system according to a second embodiment;

FIG. 10 is a drawing illustrating an examination order input screen according to the second embodiment;

FIG. 11 is a flowchart illustrating a processing procedure performed by a medical information processing system according to a third embodiment; and

FIG. 12 is a drawing illustrating an examination order input screen according to the third embodiment.

### DETAILED DESCRIPTION

A medical information processing apparatus according to an embodiment includes processing circuitry. The processing circuitry is configured to receive, with respect to each of a plurality of examinations for diagnosing a disease suspected in a subject, first information indicating whether or not it is necessary to obtain confirmation from a doctor before the examination is performed and second information indicating whether or not it is necessary to obtain consent from the subject or an appropriate representative before the examination is performed. The processing circuitry is configured to set each of the plurality of examinations with the first information and the second information as additional information. The processing circuitry is configured to control an examination order including the plurality of examinations on the basis of the additional information.

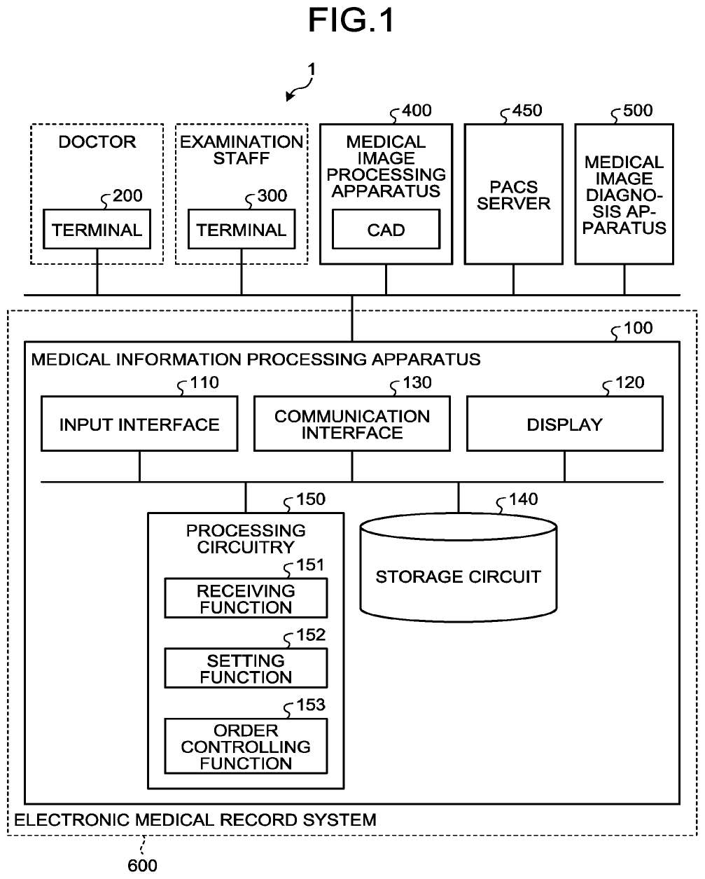

Exemplary embodiments of a medical information processing apparatus, a medical information processing system, and a medical information processing method will be explained in detail below, with reference to the accompanying drawings. In the following sections, examples of the medical information processing system including the medical information processing apparatus will be explained. Although the medical information processing system illustrated in FIG. 1 includes one each of the different types of apparatuses, the system may further include two or more apparatuses in actuality.

### First Embodiment

FIG. 1 is a diagram illustrating an exemplary configuration of a medical information processing system 1 including a medical information processing apparatus 100 according to the present embodiments. The medical information processing system 1 illustrated in FIG. 1 includes the medical information processing apparatus 100, terminals 200 and 300, a medical image processing apparatus 400, a medical image managing system (Picture Archiving and Communication System [PACS]) server 450, a medical image diagnosis apparatus 500, and an electronic medical record system 600.

The terminal 200 is used by a doctor. Examples of the terminal 200 include a Personal Computer (PC), a tablet-type PC, a Personal Digital Assistant (PDA), a mobile terminal, and the like.

The terminal 300 is used by examination staff who performs examinations. Examples of the terminal 300 include a PC, a tablet-type PC, a PDA, a mobile terminal, and the like.

The medical image diagnosis apparatus 500 is an apparatus configured to take images of an examined subject (e.g., a subject). Examples of the medical image diagnosis apparatus 500 include an X-ray diagnosis apparatus, an X-ray computer tomographic imaging apparatus (an X-ray Computed Tomography [CT] apparatus), a Magnetic Resonance Imaging (MRI) apparatus, a nuclear medicine diagnosis apparatus, and an ultrasound diagnosis apparatus.

Further, during examinations using the medical image processing apparatus 400, there are some situations in which an image processing process to detect or diagnose a disease (e.g., a lesion site) is performed, by applying a predetermined algorithm to medical image data of the subject generated by the medical image diagnosis apparatus 500. Examples of the image processing process include Computer Aided Diagnosis (CAD) processes. The medical image processing apparatus 400 has a function of implementing CAD. For example, the medical image processing apparatus 400 has installed therein a program used for implementing CAD in correspondence with each disease.

The PACS server 450 includes a database configured to store therein the medical image data generated by the medical image diagnosis apparatus 500 and a result of implementing the CAD on the medical image data. For example, the PACS server 450 receives subject information in an electronic medical record from the electronic medical record system 600 and manages the received subject information. For example, the PACS server 450 receives medical image data transmitted thereto from the medical image diagnosis apparatus 500 and receives the CAD implementation result transmitted thereto from the medical image processing apparatus 400 and manages the received medical image data and CAD implementation result so as to be kept in correspondence with the subject information.

The electronic medical record system 600 is configured to generate an electronic medical record recording subject information such as prescriptions, nursing records, and examinations provided for the subject and to store the generated electronic medical record into a storage circuit within the system.

The subject information includes basic information, diagnosis/treatment data, and examination execution information of the subject. The basic information includes a subject ID, the subject's name, birthdate, gender, blood type, height, weight, and the like. As the subject ID, identification information uniquely identifying the subject is set. The diagnosis/treatment data includes information such as numerical values (measured values) and diagnosis/treatment records, as well as recorded dates/times of the information. Examples of the diagnosis/treatment data include prescription data, nursing record data, and the like. The prescription data is data related to prescriptions and is recorded into the electronic medical record by a doctor, for example. Further, the nursing record data is data related to nursing records and is recorded into the electronic medical record by a nurse, for example. The examination execution information includes information such as examinations performed in the past and examination results of the examinations, as well as information indicating the execution dates of the examinations.

The medical information processing apparatus 100 is incorporated in the electronic medical record system 600. The medical information processing apparatus 100 is communicably connected to the terminals 200, 300, the medical image processing apparatus 400, and the like, via a network. The medical information processing apparatus 100, the terminals 200, 300, and the medical image processing apparatus 400 are connected to an intra-hospital Local Area Network (LAN) installed in a hospital, for example, and are configured to transmit information to predetermined apparatuses and to receive information transmitted thereto from predetermined apparatuses. For example, the medical information processing apparatus 100 is realized by using a computer device such as a workstation or a personal computer.

The medical information processing apparatus 100 includes an input interface 110, a display 120, a communication interface 130, a storage circuit 140, and processing circuitry 150. Possible configurations of the medical information processing apparatus 100 are not limited to the example described above; for example, the medical information processing apparatus 100 may include only the storage circuit 140 and the processing circuitry 150, so that the input interface 110, the display 120, and the communication interface 130 are used while being connected to the medical information processing apparatus 100.

The input interface 110 is connected to the processing circuitry 150 and is configured to receive operations to input various types of instructions and various types of information from an operator. More specifically, the input interface 110 is configured to convert the input operations received from the operator into electrical signals and to output the electrical signals to the processing circuitry 150. For example, the input interface 110 is realized by using a trackball, a switch button, a mouse, a keyboard, a touchpad on which input operations can be performed by touching the operation surface thereof, a touch screen in which a display screen and a touchpad are integrally formed, a contactless input circuit using an optical sensor, an audio input circuit, and/or the like. In the present embodiment, the input interface 110 does not necessarily have to include one or more physical operation component parts such as a mouse and a keyboard. For instance, possible examples of the input interface 110 include electrical signal processing circuitry configured to receive an electrical signal corresponding to an input operation from an external input device provided separately from the apparatus and to output the electrical signal to a controlling circuit.

The display 120 is connected to the processing circuitry 150 and is configured to display various types of information and various type of images. More specifically, the display 120 is configured to convert data of the various types of information and the various types of images sent thereto from the processing circuitry 150 into display-purpose electrical signals and to output the electrical signals. For example, the display 120 is realized by using a liquid crystal monitor, a Cathode Ray Tube (CRT) monitor, a touch panel, or the like.

The communication interface 130 is connected to the processing circuitry 150 and is configured to control transfer of various types of data and communication performed between the medical information processing apparatus 100 and various systems. For example, the communication interface 130 is configured to control transfer of various types of data and communication performed among the medical information processing apparatus 100, the terminal 200, and the terminal 300. For example, the communication interface 130 is realized by using a network card, a network adaptor, a Network Interface Controller (NIC), or the like.

The storage circuit 140 is connected to the processing circuitry 150 and is configured to store therein various types of information. More specifically, the storage circuit 140 is configured to store therein subject information received from various systems. For example, the storage circuit 140 is realized by using a semiconductor memory element such as a Random Access Memory (RAM) or a flash memory, or a hard disk, an optical disk, or the like. As long as the medical information processing apparatus 100 is able to access the storage circuit 140 in a network, the storage circuit 140 does not necessarily have to be built in the medical information processing apparatus 100.

The processing circuitry 150 is configured to control constituent elements of the medical information processing apparatus 100. For example, the processing circuitry 150 is configured to execute a receiving function 151, a setting function 152, and an order controlling function 153. In this situation, for example, processing functions executed by the constituent elements of the processing circuitry 150, namely, the receiving function 151, the setting function 152, and the order controlling function 153 are recorded in the storage circuit 140 in the form of computer-executable programs. The processing circuitry 150 is a processor configured to realize the functions corresponding to the programs, by reading and executing the programs from the storage circuit 140. In other words, the processing circuitry 150 that has read the programs has the functions illustrated within the processing circuitry 150 in FIG. 1. In this situation, the receiving function 151 is an example of a receiving unit. The setting function 152 is an example of a setting unit. The order controlling function 153 is an example of an order controlling unit.

The term “processor” used in the above explanations denotes, for example, a Central Processing Unit (CPU), a Graphics Processing Unit (GPU), or a circuit such as an Application Specific Integrated Circuit (ASIC) or a programmable logic device (e.g., a Simple Programmable Logic Device [SPLD], a Complex Programmable Logic Device [CPLD], or a Field Programmable Gate Array [FPGA]). The processors realize the functions by reading and executing the programs saved in the storage circuit. In this situation, instead of saving the programs in the storage circuit, it is also acceptable to directly incorporate the programs in the circuits of the processors. In that situation, the processors realize the functions by reading and executing the programs incorporated in the circuits thereof. Further, the processors in the present embodiment do not each necessarily have to be structured as a single circuit. It is also acceptable to structure one processor by combining together a plurality of independent circuits so as to realize the functions thereof.

An overall configuration of the medical information processing apparatus 100 according to the present embodiment has thus been explained. The medical information processing apparatus 100 according to the present embodiment structured as described above is configured to perform the processes described below to improve efficiency of examinations. At first, the receiving function 151 receives, with respect to each of a plurality of examinations for diagnosing a disease suspected in an examined subject (a subject), first information indicating whether or not it is necessary to obtain confirmation from a doctor before the examination is performed and second information indicating whether or not it is necessary to obtain consent from the subject or an appropriate representative before the examination is performed. Further, the setting function 152 is configured to set each of the plurality of examinations with the first information and the second information as additional information. The order controlling function 153 is configured to control an examination order including the plurality of examinations on the basis of the additional information.

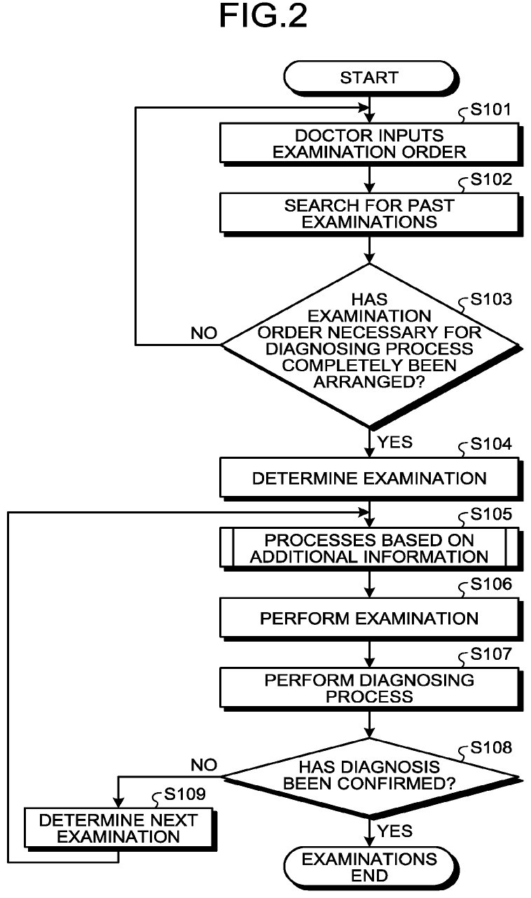

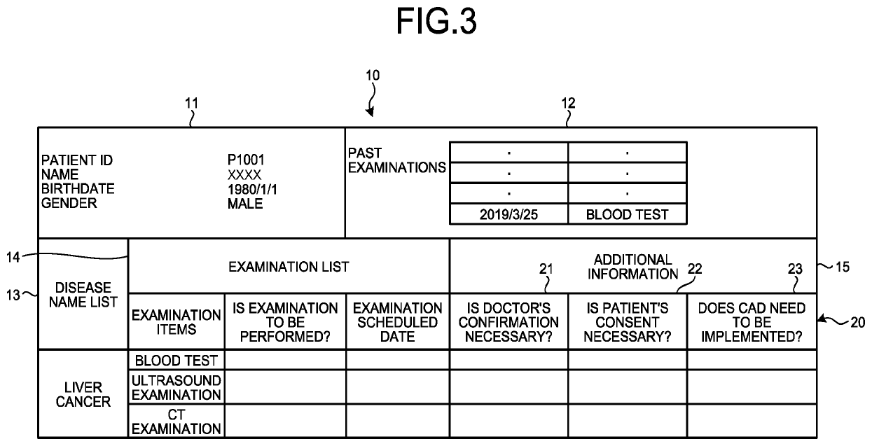

FIG. 2 is a flowchart illustrating a processing procedure performed by the medical information processing system 1 according to the first embodiment. FIGS. 3 to 6 are drawings illustrating an examination order input screen 10 according to the first embodiment. A doctor issues an examination order by operating the medical information processing apparatus 100. Alternatively, the doctor may issue the examination order by operating the terminal 200 and accessing the medical information processing apparatus 100. In the present embodiment, the latter will be explained as an example.

At step S101 in FIG. 2, the doctor inputs the examination order. More specifically, at first, the receiving function 151 of the medical information processing apparatus 100 causes a display of the terminal 200 to display the examination order input screen 10 illustrated in FIG. 3, in accordance with an operation performed by the doctor on the terminal 200. The examination order input screen 10 includes a subject information display section 11, a past examination display section 12, a disease name list display section 13, an examination list display section 14, and an additional information display section 15.

In the subject information display section 11 of the examination order input screen 10, subject information is displayed. More specifically, by operating the terminal 200, the doctor conducts a search by using a subject ID. In this situation, the receiving function 151 obtains, from the electronic medical record system 600, subject information (the subject ID, the subject's name (first and last names), the age (the birthdate), the gender, the height, the weight, the blood type, etc.) according to the subject ID. Further, as illustrated in FIG. 3, out of the obtained subject information, the receiving function 151 displays, for example, a subject ID “P1001”, the name “XXXX”, the birthdate “Jan. 1, 1980”, and the gender “male” in the subject information display section 11.

In the past examination display section 12 of the examination order input screen 10, examinations performed in the past are displayed. More specifically, the receiving function 151 obtains, from the electronic medical record system 600, examination execution information indicating the examinations performed in the past, according to the subject ID. Further, as illustrated in FIG. 3, as the obtained examination execution information, the receiving function 151 displays, for example, the examination “blood test” performed on the subject ID “P1001” most recently and the examination execution date “Mar. 25, 2019”, in the past examination display section 12.

In the disease name list display section 13 of the examination order input screen 10, one or more names of diseases (disease names) suspected in the subject are input. More specifically, the doctor refers to the result of the examination performed the last time and determines “liver cancer” as the name of the disease suspected in the subject. Further, by operating the terminal 200, the doctor inputs the disease “liver cancer” into the disease name list display section 13, as illustrated in FIG. 3. In this situation, the doctor determines the disease suspected in the subject on the basis of the result of the examination performed the last time; however, for example, the doctor may determine the disease suspected in the subject on the basis of a result of a heath checkup, a questionnaire, a referral letter, or the like. In the above example, the doctor inputs the disease “liver cancer” into the disease name list display section 13 by operating the terminal 200; however, the receiving function 151 may display, for example, a list of disease names in a pull-down menu in the disease name list display section 13, so as to have the doctor select a disease suspected in the subject from the list.

In the examination list display section 14 of the examination order input screen 10, a plurality of examination items are displayed. More specifically, the storage circuit 140 has a database in which diseases suspected in subjects are kept in correspondence with a plurality of examination items that are effective in diagnosing the diseases. For example, the database keeps “blood test”, “ultrasound examination”, and “CT examination” in correspondence, as a plurality of examinations for diagnosing the disease “liver cancer” input by the doctor. Further, when the disease “liver cancer” is displayed in the disease name list display section 13, the receiving function 151 displays the plurality of examinations “blood test”, “ultrasound examination”, and “CT examination” kept in correspondence with the disease “liver cancer” in the examination list display section 14, as illustrated in FIG. 3.

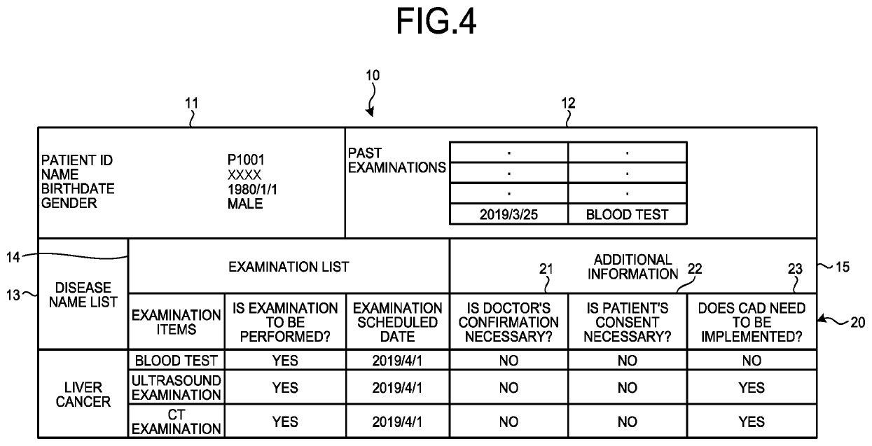

Further, with respect to each of the plurality of examinations being displayed, information indicating whether or not the examination is to be performed and information indicating an examination scheduled date are input into the examination list display section 14. More specifically, the receiving function 151 receives, with respect to each of the plurality of examinations displayed in the examination list display section 14, the information indicating whether or not the examination is to be performed and the information indicating the examination scheduled date. For example, as illustrated in FIG. 4, in accordance with operations performed by the doctor on the terminal 200, the receiving function 151 receives “Yes” as the information indicating whether or not the examination is to be performed with respect to each of the plurality of examinations “blood test”, “ultrasound examination”, and “CT examination” displayed in the examination list display section 14 and further receives “Apr. 1, 2019” as the information indicating the examination scheduled date. Further, although not illustrated in the drawings, the receiving function 151 receives information indicating an examination condition, with respect to each of the plurality of examinations displayed in the examination list display section 14. In the present example, the doctor determines the examination condition; however, in some situations, the examination staff may determine the examination condition when performing the examination. As explained herein, the medical information processing apparatus 100 according to the first embodiment is able to order the plurality of examinations altogether at once for the single subject.

In some situations, the doctor may refer to results of an already-performed examination, before determining whether or not a next examination is to be performed. In those situations, for example, by operating the terminal 200, the doctor inputs “undetermined” as information indicating whether or not the examination is to be performed with respect to the examination displayed in the examination list display section 14, so that the receiving function 151 receives the input information. Further, in some situations, the doctor may refer to results of an already-performed examination, before determining the condition of the next examination. In those situations, for example, by operating the terminal 200, the doctor inputs “undetermined” as information indicating the examination condition with respect to the examination displayed in the examination list display section 14, so that the receiving function 151 receives the input information. As explained herein, by using the medical information processing apparatus 100 according to the first embodiment, it is possible to order the examination in the undetermined state, even when the examination is determined in accordance with results of an already-performed examination.

With respect to each of the plurality of examinations displayed in the examination list display section 14, the following pieces of information are input in the additional information display section 15 of the examination order input screen 10: first information 21 indicating whether or not it is necessary to obtain confirmation from the doctor before the examination is performed; second information 22 indicating whether or not it is necessary to obtain consent from the subject or an appropriate representative before the examination is performed; and third information 23 indicating whether or not it is necessary to implement CAD. More specifically, the receiving function 151 receives, via the additional information display section 15, the first information 21, the second information 22, and the third information 23, with respect to each of the plurality of examinations “blood test”, “ultrasound examination”, and “CT examination” displayed in the examination list display section 14. In this situation, examples of the examinations for which it is necessary to obtain confirmation from a doctor include examinations that are highly invasive for the subjects, such as a CT examination, a biopsy, and the like. Examples of the examinations for which it is necessary to obtain consent from the subject or an appropriate representative include examinations that involve side effects and/or precautions for the subjects such as a contrast CT examination, an endoscopy examination, and the like. Further, the appropriate representative may be a spouse, a parent, or the like.

For example, as illustrated in FIG. 4, in accordance with operations performed by the doctor on the terminal 200, with respect to the examination “blood test” displayed in the examination list display section 14, the receiving function 151 receives “No” indicating that it is not necessary to obtain confirmation from a doctor before the examination is performed as the first information 21 and receives “No” indicating that it is not necessary to obtain consent from the subject or an appropriate representative before the examination is performed as the second information 22. In this situation, because it is not necessary to implement CAD for blood tests, “No” indicating that it is not necessary to implement CAD is set as the third information 23 for the examination “blood test”. Further, in accordance with operations performed by the doctor on the terminal 200, with respect to each of the examinations “ultrasound examination” and “CT examination” displayed in the examination list display section 14, the receiving function 151 receives “No” as the first information 21, “No” as the second information 22, and “Yes” indicating that it is necessary to implement CAD as the third information 23.

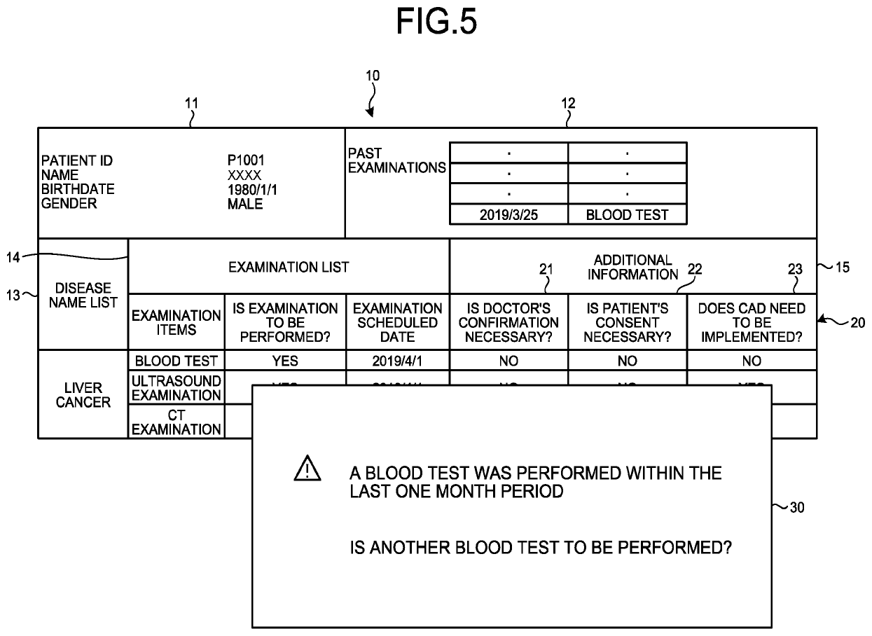

At step S102 in FIG. 2, a search is conducted for past examinations. More specifically, with respect to the plurality of examinations “blood test”, “ultrasound examination”, and “CT examination” displayed in the examination list display section 14, the order controlling function 153 at first judges whether or not there is an examination that was performed within a predetermined time period. For example, in the past examination display section 12 of the examination order input screen 10, the examination “blood test” performed most recently and the examination execution date “Mar. 25, 2019” are displayed as the examination execution information of the examinations performed in the past. For example, when the predetermined time period is one month, among the plurality of examinations “blood test”, “ultrasound examination”, and “CT examination” displayed in the examination list display section 14, “blood test” is an examination that was performed within the predetermined time period. In that situation, by using a pop-up screen 30 illustrated in FIG. 5, the order controlling function 153 displays, on the examination order input screen 10, information prompting the doctor to confirm whether or not the examination “blood test”, which was performed within the predetermined time period, is to be performed. For example, the pop-up screen 30 displays a message such as “A blood test was performed within the last one month period. Are you going to order another blood test?”.

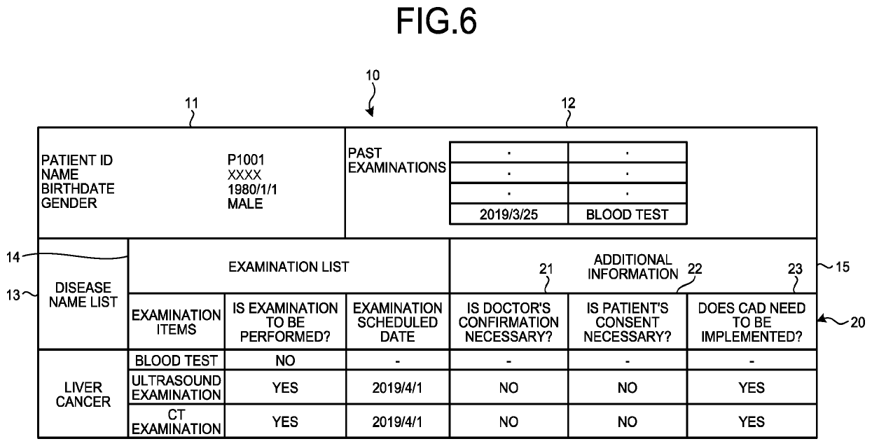

Further, for example, when having received a cancellation instruction in response to the message via the terminal 200 of the doctor, the receiving function 151 cancels the examination “blood test” that was performed within the predetermined time period. In that situation, as illustrated in FIG. 6, the receiving function 151 changes the information indicating whether or not the examination is to be performed from “Yes” to “No”, with respect to the examination “blood test” displayed in the examination list display section 14. Further, for example, with respect to the examination “blood test” displayed in the examination list display section 14, the receiving function 151 nullifies the information indicating the examination scheduled date and the information received as the first information 21, the second information 22, and the third information 23. As for examinations that were performed earlier than the predetermined number of days ago, the nullification is not performed even though the examinations were performed in the past. Further, as for the examination that was performed within the predetermined time period, the receiving function 151 may automatically cancel the examination without obtaining the confirmation from the doctor. As explained herein, when there is an examination item that was performed within the predetermined time period, the medical information processing apparatus 100 according to the first embodiment is able to eliminate the examination from the examination list.

At step S103 in FIG. 2, it is judged whether or not an examination order that is necessary for a diagnosing process has completely been arranged. For example, the receiving function 151 performs a process of asking the doctor whether or not an examination order necessary for the diagnosing process has completely been arranged. As a judgment result, when the doctor determines that the examination order necessary for the diagnosing process has not completely been arranged (step S103: No), the processes at step S101 and thereafter are performed, because there is a change or addition to the examination items. On the contrary, as a judgment result, when the doctor determines that the examination order necessary for the diagnosing process has completely been arranged (step S103: Yes), the doctor issues an input completion instruction by operating the terminal 200, for example, because there is no change or addition to the examination items. In that situation, according to the input completion instruction received via the terminal 200, the setting function 152 sets each of the plurality of examinations “ultrasound examination” and “CT examination” displayed in the examination list display section 14, with the first information 21 “No”, the second information 22 “No”, and the third information 23 “Yes” received via the additional information display section 15, as additional information 20.

In the present example, the receiving function 151 asks the doctor whether or not the examination order necessary for the diagnosing process has completely been arranged; however, possible embodiments are not limited to this example. For instance, when the receiving function 151 determines that the examination order necessary for the diagnosing process has completely been arranged, the setting function 152 may automatically set each of the plurality of examinations “ultrasound examination” and “CT examination” displayed in the examination list display section 14, with the first information 21 “No”, the second information 22 “No”, and the third information 23 “Yes” received via the additional information display section 15, as the additional information 20. As explained herein, the medical information processing apparatus 100 according to the first embodiment is able to present, on the examination order input screen 10 at the initial stage, the plurality of examination items that are effective in diagnosing the disease, so that the doctor is able to determine, on the examination order input screen 10 at the ordering stage, the examinations to be performed among the plurality of examinations.

At step S104 in FIG. 2, an examination to be performed is determined. In one example, from among the plurality of examinations arranged in a sequential order in the examination list, the examination listed first is determined as the examination to be performed. Possible methods for determining the examination to be performed are not limited to the method in this example. It is acceptable to determine the examination to be performed by using the method described below.

For example, the order controlling function 153 has, at all times, understanding of appointment statuses of examinations and current waiting time periods within the electronic medical record system 600. Accordingly, when the examination order including the plurality of examinations has completely been arranged, the order controlling function 153 automatically determines a sequential order of the plurality of examinations on the basis of a schedule of the apparatuses (e.g., the medical image diagnosis apparatus 500 and the medical image processing apparatus 400) that perform the examinations and a schedule of the examination staff.

Further, among the plurality of examinations, when there is an examination that cannot be performed at the current point in time according to the schedule of the apparatuses and the schedule of the examination staff, the order controlling function 153 outputs information prompting the doctor to confirm the examination that cannot be performed at the current point in time, to the terminal 200 of the doctor. In this situation, when having received a switch instruction from the terminal 200 of the doctor in response to the information, the order controlling function 153 determines a sequential order of the plurality of examinations on the basis of the schedule of the apparatuses and the schedule of the examination staff. In this manner, the medical information processing apparatus 100 according to the first embodiment is able to designate the sequential order of the plurality of examinations.

In the present example, the examination that cannot be performed at the current point in time is an examination having no vacancy in the schedule, as a result of taking the appointment statuses of the examinations and the current waiting time periods into consideration; however, possible embodiments are not limited to this example. For instance, the examination that cannot be performed at the current point in time may refer to the situation in which it is determined whether or not the next examination is to be performed depending on results of an already-performed examination. In other words, the examination that cannot be performed at the current point in time may refer to the situation in which the additional information 20 is set as “undetermined” as the information indicating whether or not the examination is to be performed. In the present embodiment, for example, it is possible to order an examination in an undetermined state, even when the examination is to be determined in accordance with results of an already-performed examination. It is also possible to order an examination in an undetermined state, even when the examination condition (explained later) of the examination has not yet been determined. As explained herein, the medical information processing apparatus 100 according to the first embodiment is able to order the examination, even when the examination cannot be performed at the current point in time.

When the sequential order of the plurality of examinations has been determined, the order controlling function 153 outputs the examination order including the plurality of examinations to various systems in the hospital, in accordance with an operation performed by the doctor on the terminal 200. Examples of the various systems include a Hospital Information System (HIS), a radiology department information management system such as a Radiology Information System (RIS), and a medical image management system such as a Picture Archiving and Communication System (PACS). As a result, appointments are made for the plurality of examinations. Further, by performing the process described below, the order controlling function 153 controls the examination order including the plurality of examinations, on the basis of the additional information 20 set with each of the plurality of examinations.

At step S105 in FIG. 2, processes based on the additional information are performed. More specifically, with respect to the examination to be performed, the order controlling function 153 checks to see whether or not “No” is set as the first information 21 while “No” is also set as the second information 22, in the additional information 20. For instance, let us discuss an example in which “No” is set as the first information 21 and also “No” is set as the second information 22, with respect to the examination to be performed. In that situation, the order controlling function 153 outputs the examination order related to the examination to be performed to the terminal 300 of the examination staff. The examination order related to the examination to be performed includes the subject information and the examination item to be performed. Subsequently, step S106 (explained later) will be performed.

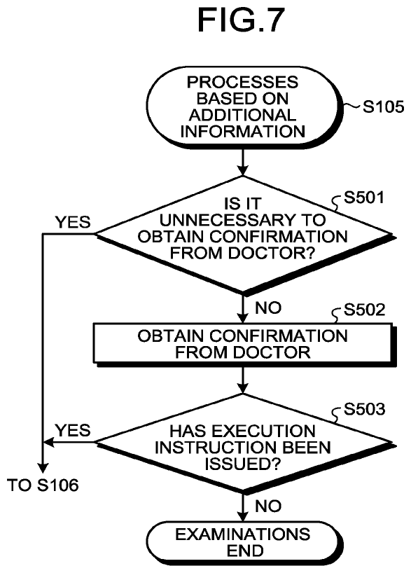

FIG. 7 is a flowchart illustrating an example of the processes based on the additional information. For instance, let us discuss an example in which, with respect to an examination to be performed, “Yes” is set as the first information 21, to indicate that it is necessary to obtain confirmation from the doctor before the examination is performed. In other words, it is indicated that, for the examination, it is necessary to obtain confirmation from the doctor before the examination is performed (step S501: No). In that situation, the order controlling function 153 outputs information prompting the doctor to confirm before the examination is performed, to the terminal 200 of the doctor (step S502). When having received an instruction to perform the examination from the terminal 200 of the doctor in response to the information (step S503: Yes), the order controlling function 153 outputs the examination order related to the examination to be performed, to the terminal 300 of the examination staff. After that, step S106 (explained later) will be performed. On the contrary, for example, when the order controlling function 153 receives an instruction not to perform the examination from the terminal 200 of the doctor in response to the information (step S503: No), the examinations end.

Let us discuss another example in which, with respect to an examination to be performed, “undetermined” is set as the information indicating whether or not the examination is to be performed. In other words, it is indicated that whether or not the examination is to be performed is determined on the basis of results of an already-performed examination. In that situation also, for the examination, because it is necessary to obtain confirmation from the doctor before the examination is performed, the processes at step S502 and thereafter will be performed.

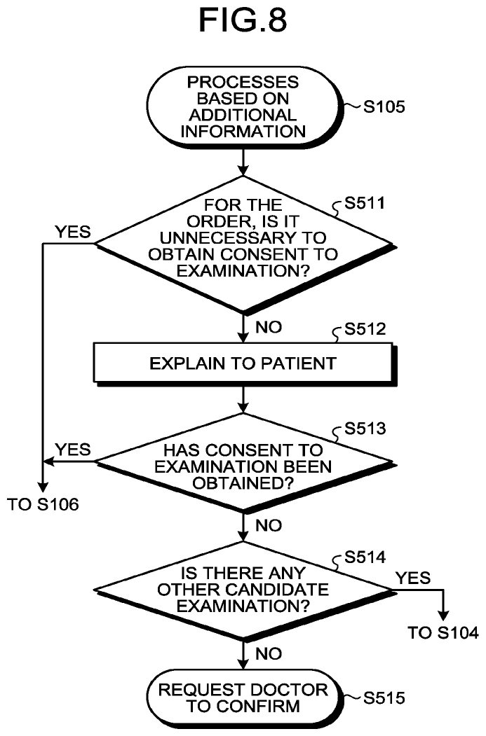

FIG. 8 is a flowchart illustrating another example of the processes based on the additional information. For instance, let us discuss an example in which, with respect to the examination to be performed, “Yes” is set as the second information 22, to indicate that it is necessary to obtain consent from the subject or an appropriate representative before the examination is performed. In other words, it is indicated that, for the examination, it is necessary to obtain consent from the subject or an appropriate representative before the examination is performed (step S511: No). In that situation, the order controlling function 153 outputs, to the terminal 200 of the doctor, information notifying the doctor that it is necessary to obtain consent from the subject or an appropriate representative before the examination is performed (step S512). In this situation, the order controlling function 153 outputs, to an output device such as a printer, a consent form used for obtaining the consent from the subject or an appropriate representative before the examination is performed. In this situation, when having received the information via the terminal 200 of his/her own, the doctor explains about the examination to the subject or an appropriate representative, and let us assume that the doctor has obtained the consent from the subject or the appropriate representative by using the consent form. Further, when having received consent information indicating that the consent has been obtained from the subject or an appropriate representative, from the terminal 200 of the doctor in response to the information (step S513: Yes), the order controlling function 153 outputs the examination order related to the examination to be performed, to the terminal 300 of the examination staff. After that, step S106 (explained later) will be performed. On the contrary, when the doctor explains about the examination to the subject or an appropriate representative, but is unable to obtain the consent from the subject or the appropriate representative, e.g., when the order controlling function 153 has received, from the terminal 200 of the doctor, information indicating that it is not possible to obtain the consent from the subject or an appropriate representative (step S513: No), and when there is another candidate examination (step S514: Yes), step S104 will be performed. On the contrary, when there is no other candidate examination (step S514: No), the order controlling function 153 outputs, to the terminal 200 of the doctor, information prompting the doctor to confirm that there is no other candidate examination (step S515).

At step S106 in FIG. 2, the examination is performed. More specifically, when an examination condition is set with the examination to be performed, the examination is performed under the set examination condition. On the contrary, when no examination condition is set with the examination to be performed, the examination is performed under an examination condition created in a manual in the hospital. In this situation, with respect to the examination to be performed, when “undetermined” is set as the information indicating the examination condition, the process described below will be performed.

For instance, let us discuss an example in which the examination to be performed is an examination using the medical image diagnosis apparatus 500, and “undetermined” is set as the information indicating the examination condition of the examination to be performed. In that situation, the examination staff operates the terminal 300 and outputs, to the terminal 200 of the doctor, information indicating that an examination condition needs to be determined by the doctor. The information includes the results of the examinations that have so far been performed. The doctor refers to the information received via the terminal 200, determines an examination condition, and outputs the determined examination condition to the terminal 300 of the examination staff by operating the terminal 200. After that, the examination staff performs the examination on the subject under the examination condition determined by the doctor, by using the medical image diagnosis apparatus 500.

Further, let us discuss another example in which the examination to be performed is an examination using the medical image diagnosis apparatus 500, while “undetermined” is set as the information indicating the examination condition of the examination to be performed, and the examination staff has determined an examination condition. In that situation, the examination staff operates the terminal 300 and outputs, to the terminal 200 of the doctor, information indicating that the determined examination condition needs to be confirmed by the doctor. The information includes the results of the examinations that have so far been performed. When the terminal 300 of the examination staff receives, from the terminal 200 of the doctor, an instruction to perform the examination in response to the information, the examination staff performs the examination on the subject under the examination condition determined by the doctor, by using the medical image diagnosis apparatus 500.

At step S107 in FIG. 2, a diagnosing process is performed. For instance, let us discuss an example in which the performed examination was an examination using the medical image diagnosis apparatus 500, and “Yes” is set with the performed examination as the third information to indicate that it is necessary to implement CAD. In other words, the third information “Yes” indicates that it is necessary to implement CAD on the medical image data acquired in the performed examination. In this situation, the order controlling function 153 instructs the medical image diagnosis apparatus 500 to transmit the medical image data of the subject generated by the medical image diagnosis apparatus 500 to the medical image processing apparatus 400 via the network. Further, the order controlling function 153 instructs the medical image processing apparatus 400 to implement an automatic diagnosing process using CAD corresponding to the disease “liver cancer” suspected in the subject, on the medical image data of the subject generated by the medical image diagnosis apparatus 500. A result of the automatic diagnosing process using CAD is, for example, output from either the medical image processing apparatus 400 or the terminal 300 of the examination staff to the medical information processing apparatus 100, as an examination result. As explained herein, the order controlling function 153 is able to cause the automatic diagnosing process using CAD to be performed while using the medical image data, by outputting the examination order set with the third information “Yes” as the additional information 20. In this situation, the diagnosing process using the medical image data may automatically be performed as the automatic diagnosing process or may be performed by a doctor.

In this situation, when the performed examination was an examination using a numerical value such as a blood test, while the third information “No” indicates that it is not necessary to implement CAD, a diagnosing process is performed by checking to see whether or not the numerical value falls within a predetermined range. A result of the diagnosing process is, for example, output from the terminal 300 of the examination staff to the medical information processing apparatus 100 as an examination result. In this situation, the diagnosing process may automatically be performed or may be performed by a doctor.

At step S108 in FIG. 2, it is judged whether or not a diagnosis has been confirmed. More specifically, the order controlling function 153 receives the examination result output from either the medical image processing apparatus 400 or the terminal 300 of the examination staff and checks to see whether or not the examination result confirms the disease suspected in the subject. For example, when the examination result does not confirm the disease suspected in the subject, the order controlling function 153 determines that the diagnosis has not been confirmed by the result of the performed examination alone (step S108: No). In that situation, step S109 will be performed.

At step S109 in FIG. 2, the next examination is determined. More specifically, from among the plurality of examinations, the next examination following the performed examination is determined, for example. Subsequently, the processes at step S105 and thereafter will be performed.

At step S109, on the basis of the results of the performed examination, the order controlling function 153 may change the sequential order of the examinations that have not yet been performed (hereinafter, “unperformed examinations”), while prioritizing an examination that will increase the possibility of detecting the disease. For example, when it is desirable to confirm the diagnosis for the convenience of the subject or the like, performing a biopsy instead of performing a diagnosing process with images may increase the possibility of detecting a disease in some situations. In those situations, the order controlling function 153 outputs, to the terminal 200 of the doctor, information prompting the doctor to confirm whether or not an examination that will increase the possibility of detecting the disease is to be performed. In this situation, when having received a change instruction from the terminal 200 of the doctor in response to the information, the order controlling function 153 changes the sequential order of the unperformed examinations. As explained herein, the medical information processing apparatus 100 according to the first embodiment is able to prioritize the examination that will increase the possibility of detecting the disease.

In this situation, the order controlling function 153 may change the sequential order of the unperformed examinations, on the basis of the schedule of the apparatuses (e.g., the medical image diagnosis apparatus 500 and the medical image processing apparatus 400) that perform the examination and the schedule of the examination staff. In that situation, the order controlling function 153 outputs, to the terminal 200 of the doctor, information prompting the doctor to confirm. In this situation, when having received a change instruction from the terminal 200 of the doctor in response to the information, the order controlling function 153 changes the sequential order of the unperformed examinations on the basis of the schedule of the apparatuses and the schedule of the examination staff. As explained herein, the medical information processing apparatus 100 according to the first embodiment is able to prioritize examinations that can be performed earlier.

Further, at step S109, on the basis of the results of the performed examination, it is acceptable to cancel an examination having a lower possibility of detecting the disease among the unperformed examinations. More specifically, the order controlling function 153 outputs, to the terminal 200 of the doctor, information prompting the doctor to confirm whether or not the examination having a lower possibility of detecting the disease is to be performed. In this situation, when having received a cancellation instruction from the terminal 200 of the doctor in response to the information, the order controlling function 153 cancels the examination having a lower possibility of detecting the disease. As explained herein, when there is an examination having a lower possibility of detecting the disease on the basis of the results of the performed examination, the medical information processing apparatus 100 according to the first embodiment is able to eliminate the examination from the examination list.

For example, when the examination results have confirmed the disease suspected in the subject, the order controlling function 153 determines that the diagnosis has been confirmed by the results of the performed examination (step S108: Yes), and the examinations end. In this situation, the setting function 152 may cancel the unperformed examinations. More specifically, the order controlling function 153 outputs, to the terminal 200 of the doctor, information prompting the doctor to confirm whether or not the unperformed examinations are to be performed. In this situation, when having received a cancellation instruction from the terminal 200 of the doctor in response to the information, the setting function 152 cancels the unperformed examinations. As explained herein, when the disease suspected in the subject is discovered on the basis of the results of the performed examination at the stage where there are unperformed examinations, the medical information processing apparatus 100 according to the first embodiment does not need to perform the unperformed examinations.

As explained above, with respect to each of the plurality of examinations presented in the examination order, the medical information processing apparatus 100 according to the first embodiment is configured to set, as the additional information, whether or not confirmation from the doctor is necessary and whether or not consent from the subject or an appropriate representative is necessary. For example, in the first embodiment, by operating the terminal 200, the doctor sets each of all the scheduled examinations with the first information 21 “No”, the second information 22 “No”, and the third information 23 “Yes”, as the additional information 20. Accordingly, the medical information processing apparatus 100 according to the first embodiment is able to order the plurality of examinations altogether at once for the single subject and is thus able to shorten the order issuance time period between the performed examination and the next examination. In other words, the burden imposed on the doctor, the examination staff, the subject, and the like is reduced. As explained above, the medical information processing apparatus 100 according to the first embodiment is able to improve efficiency of the examinations.

To further improve efficiency of the examinations, the medical information processing apparatus 100 according to the first embodiment may, for example, arrange a mobile terminal to be borrowed by the subject so that, when an examination is to be performed on the subject, the order controlling function 153 transmits guidance indicating the reception location of the examination or the like to the mobile terminal, so as to be displayed on the display of the mobile terminal.

### Second Embodiment

For the medical information processing apparatus 100 according to a second embodiment, an example will be explained in which a disease other than the disease suspected in the subject is discovered on the basis of the results of the performed examination. In the second embodiment, the doctor determines, in advance, a procedure to be taken when a candidate for the disease is changed or when the unperformed examinations among the plurality of examinations are not to be performed.

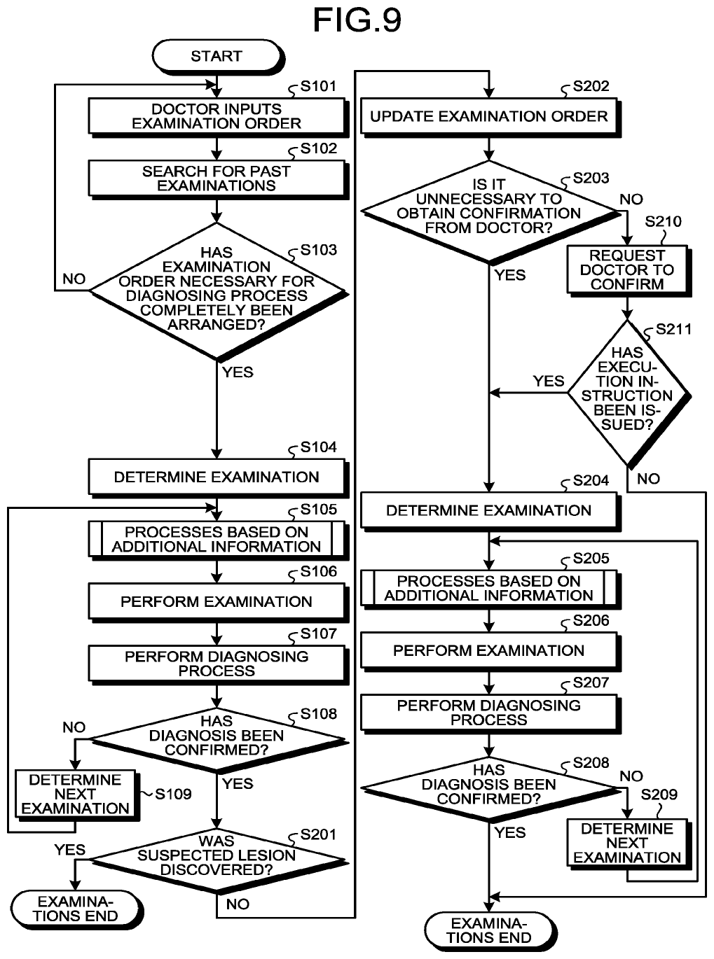

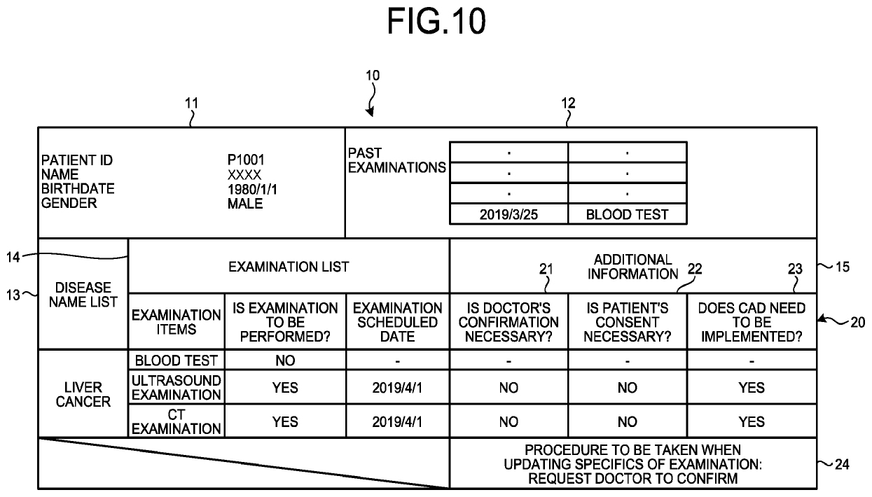

FIG. 9 is a flowchart illustrating a processing procedure performed by the medical information processing system 1 according to the second embodiment. FIG. 10 is a drawing illustrating the examination order input screen 10 according to the second embodiment.

At first, in FIG. 9, steps S101 through S103 are performed. In this situation, for example, at step S103, the receiving function 151 receives, via the additional information display section 15, fourth information 24 indicating that it is necessary to obtain confirmation from the doctor when a candidate for the disease is changed or when unperformed examinations among the plurality of examinations are not to be performed. For example, as illustrated in FIG. 10, the receiving function 151 receives “Procedure to be taken when updating specifics of an examination: Request the doctor to confirm” as the fourth information 24, through an operation performed by the doctor on the terminal 200.

Further, at step S103, when there is no change or addition to the examination items, the setting function 152 sets the plurality of examinations displayed as an examination order in the examination list display section 14 with the first information 21, the second information 22, and the third information 23 received via the additional information display section 15, as additional information 20. Further, the setting function 152 sets the fourth information 24 received via the additional information display section 15 as additional information 20.

Subsequently, in FIG. 9, steps S104 through S109 are performed. In the second embodiment, at step S106, regarding the automatic diagnosing process using CAD, not only the CAD corresponding to the disease designated in advance, but also CAD corresponding to the other disease besides the designated disease is also applied in parallel.

For example, at step S108 in FIG. 9, it is judged whether or not a diagnosis has been confirmed. More specifically, the order controlling function 153 receives the examination results output from either the medical image processing apparatus 400 or the terminal 300 of the examination staff and further checks to see whether or not the examination results have confirmed the disease. For example, when the examination results have not confirmed the disease, the order controlling function 153 determines that the diagnosis has not been confirmed by the results of the performed examination alone (step S108: No). In this situation, step S109 will be performed. In the contrary example, when the examination results have confirmed the disease, the order controlling function 153 determines that the diagnosis has been confirmed by the results of the performed examination (step S108: Yes). In this situation, step S201 will be performed.

At step S201 in FIG. 9, it is judged whether or not the disease (a lesion) suspected in the subject has been discovered. Let us discuss an example in which, as a result of the automatic diagnosing process using CAD corresponding to the disease “liver cancer” designated in advance, the disease “liver cancer” suspected in the subject has been discovered. In this situation, because the lesion suspected in the subject has been discovered (step S201: Yes), the examinations end. Let us discuss another example in which the disease “liver cancer” suspected in the subject was not discovered on the basis of the results of the automatic diagnosing process using CAD corresponding to the disease “liver cancer”, but a disease other than the disease “liver cancer” suspected in the subject was discovered on the basis of the results of the automatic diagnosing process using CAD corresponding to the disease “lung cancer”. In this situation, because the lesion other than the lesion suspected in the subject was discovered (step S201: No), step S202 will be performed.

At step S202 in FIG. 9, the examination order is updated. More specifically, by referring to the database in the storage circuit 140 keeping the diseases in correspondence with the plurality of examination items, the order controlling function 153 searches for a plurality of examinations for diagnosing the disease “lung cancer”. Further, the order controlling function 153 changes the candidate for the disease from “liver cancer” to “lung cancer” and also changes the unperformed examinations among the plurality of examinations, by making a list of examinations that are not duplicate of the performed examinations, from among the plurality of examinations corresponding to the disease “lung cancer”. In other words, the examination order is updated.

At step S203 in FIG. 9, it is judged whether or not it is unnecessary to obtain confirmation from the doctor. More specifically, the order controlling function 153 checks to see whether or not the examination order is set with the fourth information 24 “Procedure to be taken when updating specifics of an examination: Request the doctor to confirm”, as additional information 20. In other words, it is checked to see whether or not confirmation from the doctor is unnecessary. When the confirmation from the doctor is unnecessary (step S203: Yes), steps S204 through S209 in FIG. 9 are performed, as the same processes as those at steps S104 through S109 in FIG. 2.

On the contrary, when the confirmation from the doctor is necessary (step S203: No), the order controlling function 153 outputs, to the terminal 200 of the doctor, information prompting the doctor to confirm either that the candidate for the disease has been changed or that the unperformed examinations among the plurality of examinations are not to be performed (step S210). For example, the post-change examinations are not set with the first information 21, the second information 22, and the third information 23, as the additional information 20. In that situation, the order controlling function 153 also outputs, to the terminal 200 of the doctor, information notifying the doctor that the post-change examinations are not set with the first information 21, the second information 22, and the third information 23. In this situation, as a result of performing the same process as the process at step S101 in the first embodiment, the post-change examinations are set with the first information 21, the second information 22, and the third information 23. Among the post-change examinations, when there are one or more examinations of which the results have already been received, it is possible to omit the process of prompting the doctor to confirm. In this situation, when the order controlling function 153 has received an instruction to perform the examinations from the terminal 200 of the doctor in response to the information, for example, steps S204 through S209 in FIG. 9 will be performed.

As explained above, in the medical information processing apparatus 100 according to the second embodiment, the receiving function 151 is configured to receive the fourth information 24 indicating that it is necessary to obtain confirmation from the doctor when the candidate for the disease has been changed or when the unperformed examinations among the plurality of examinations are not to be performed on the basis of the results of the performed examination. The setting function 152 is configured to set the examination order with the fourth information 24 as the additional information 20. Further, when the candidate for the disease has been changed or when the unperformed examinations are not to be performed, the order controlling function 153 is configured to output the information prompting the doctor to confirm, on the basis of the fourth information 24. As explained herein, with the medical information processing apparatus 100 according to the second embodiment, because the doctor determines, in advance, the procedure to be taken when the candidate for the disease has been changed or when the unperformed examinations are not to be performed, it is possible to improve efficiency of the examinations.

In addition, to further improve efficiency of the examinations, the medical information processing apparatus 100 according to the second embodiment may be configured so that, for example, the order controlling function 153 transmits, to the terminal 200 of the doctor, a message related to a referral to another specialist so as to be displayed on the display of the terminal 200, when the order controlling function 153 determines at step S203 that it is desirable to make the referral to another department or another hospital depending on the type of the lesion.

### Third Embodiment

Regarding the medical information processing apparatus 100 according to a third embodiment, an example will be explained in which results of an image processing process using CAD are defective. In the third embodiment, the doctor determines, in advance, a procedure to be taken when results of an image processing process are defective.

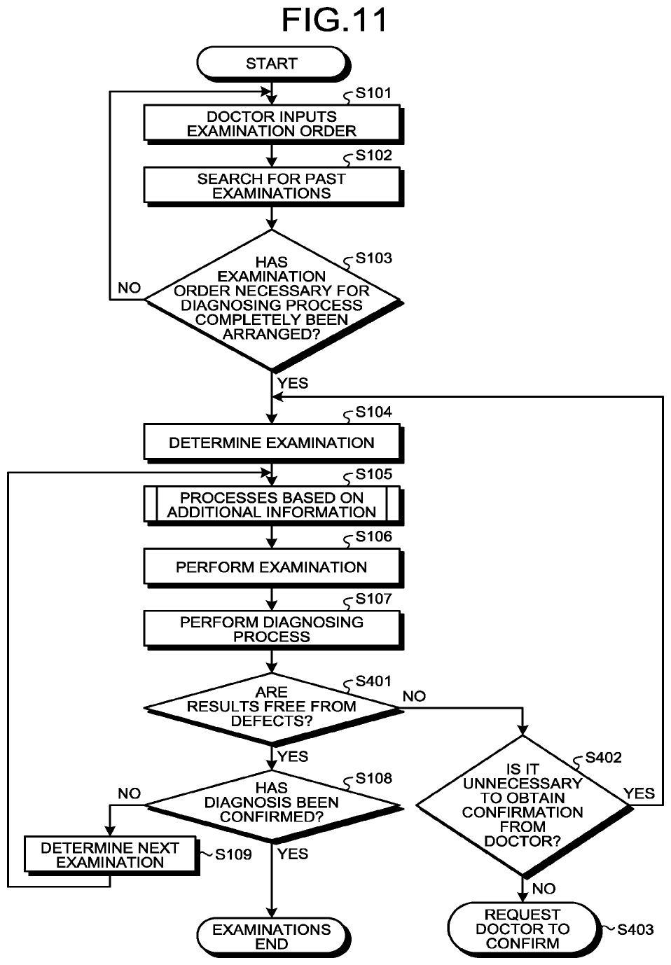

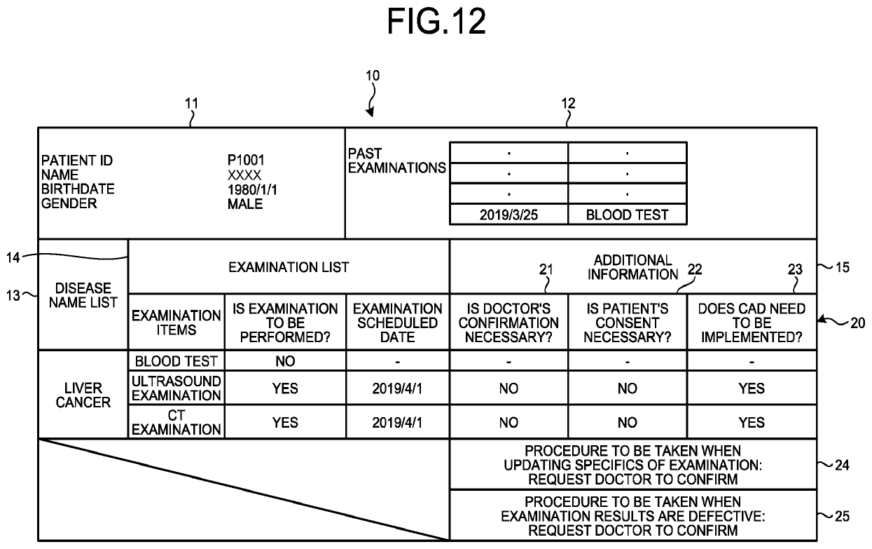

FIG. 11 is a flowchart illustrating a processing procedure performed by the medical information processing system 1 according to the third embodiment. FIG. 12 is a drawing illustrating the examination order input screen 10 according to the third embodiment.

At first, in FIG. 11, steps S101 through S103 are performed. In this situation, for example, at step S103, the receiving function 151 receives, via the additional information display section 15, fifth information 25 indicating that it is necessary to obtain confirmation from the doctor when results of an image processing process are defective. For example, as illustrated in FIG. 12, the receiving function 151 receives “Procedure to be taken when examination results are defective: Request the doctor to confirm” as the fifth information 25, through an operation performed by the doctor on the terminal 200.

Further, at step S103, when there is no change or addition to the examination items, the setting function 152 sets the plurality of examinations displayed as an examination order in the examination list display section 14 with the first information 21, the second information 22, and the third information 23 received via the additional information display section 15, as additional information 20. Further, the setting function 152 sets the fourth information 24 and the fifth information 25 received via the additional information display section 15 as additional information 20.

Subsequently, in FIG. 11, steps S104 through S107 are performed. In this situation, the order controlling function 153 checks to see whether or not the results of the automatic diagnosing process using CAD are defective (step S401). For example, at step S106, the automatic diagnosing process using CAD is performed on the medical image data of the subject generated by the medical image diagnosis apparatus 500, and let us discuss the situation where the examination results are defective. Examples of the situation where the examination results are defective include when the examination condition is not satisfied and when it was impossible to implement CAD due to an error. Examples of the situation where the examination condition is not satisfied include when the subject failed to hold his/her breath while the image was taken.

In the situation where the results of the automatic diagnosing process using CAD are free from defects (step S401: Yes), when the disease suspected in the subject is discovered on the basis the results of the performed examination, the order controlling function 153 confirms the diagnosis (step S108: Yes), and the examinations end.

In the situation where the results of the automatic diagnosing process using CAD are not free from defects (step S401: No), the order controlling function 153 checks to see whether or not the examination order is set with the fifth information 25 “Procedure to be taken when examination results are defective: Request the doctor to confirm”, as additional information 20. In other words, the order controlling function 153 checks to see whether or not it is unnecessary to obtain confirmation from the doctor (step S402).

Let us discuss an example in which the examination order is set with the fifth information 25, and the confirmation from the doctor is unnecessary (step S402: Yes). In this situation, the next examination to be performed is set with “Yes” as the information indicating whether or not the examination is to be performed. In that situation, the processes at step S104 and thereafter will be performed.

Let us discuss another example in which the examination order is not set with the fifth information 25, and the confirmation from the doctor is necessary (step S402: No). In this situation, the order controlling function 153 outputs, to the terminal 200 of the doctor, information notifying the doctor that the results of the automatic diagnosing process using CAD are defective and prompting the doctor to confirm (step S403). In this situation, the doctor may change the examination condition (e.g., the image taking condition of the images) of the performed examination by using the terminal 200 and may transmit a re-examination instruction so that the examination is performed again by using the post-change examination condition. In that situation, when the order controlling function 153 receives the re-examination instruction from the terminal 200 of the doctor in response to the information, the processes at step S105 and thereafter are performed, for example, so that the examination is performed under the post-change examination condition. Further, the doctor may transmit a next examination execution instruction indicating that the next examination following the performed examination should be performed. In that situation, when the order controlling function 153 receives the next examination execution instruction from the terminal 200 of the doctor in response to the information, the processes at step S104 and thereafter are performed, for example, so that the next examination is performed under the determined examination condition.

Let us discuss yet another example in which the examination order is set with the fifth information 25, and it is unnecessary to obtain confirmation from the doctor (step S402: Yes), but the next examination to be performed is set with “undetermined” as the information indicating whether or not the examination is to be performed. In that situation, the order controlling function 153 outputs, to the terminal 200 of the doctor, information prompting the doctor to confirm before the examination is performed. In this situation, when the order controlling function 153 receives the examination execution instruction from the terminal 200 of the doctor in response to the information, the processes at step S104 and thereafter are performed, for example, so that the next examination is performed under the determined examination condition.

As explained above, in the medical information processing apparatus 100 according to the third embodiment, the receiving function 151 is configured to receive the fifth information 25 indicating that it is necessary to obtain confirmation from the doctor when results of an image processing process are defective. The setting function 152 is configured to set the examination order with the fifth information 25 as the additional information 20. Further, when results of an image processing process are defective, the order controlling function 153 is configured to output the information prompting the doctor to confirm, on the basis of the fifth information 25. As explained herein, with the medical information processing apparatus 100 according to the third embodiment, because the doctor determines, in advance, the procedure to be taken when results of an image processing process are defective, it is possible to improve efficiency of the examinations.

### Other Modification Examples

Possible embodiments are not limited to the embodiments described above. For instance, at step S105, when an examination to be performed requires confirmation from the doctor or consent from the subject, the destination to which the examination order is sent may be changed to a different doctor or a different hospital. For example, there may be some situations where it is impossible to perform the examination at the hospital but the case is urgent or where there is no vacancy in the schedule of the apparatuses performing the examination or the schedule of the examination staff but the case is urgent. In those situations, the doctor in charge or the hospital may be changed to a different one.

Further, in any of the embodiments, the hospital issuing an examination order may be different from the hospital performing the examinations. For example, when a first hospital issuing an examination order is unable to perform one or more of the examinations in the examination order, but a second hospital is able to perform the one or more examinations, the examinations may be performed by a collaboration between the first hospital and the second hospital.

Further, in any of the embodiments, the order controlling function 153 may confirm the diagnosis or may determine the next examination, by using Artificial Intelligence (AI) learning or machine learning based on a neural network. For example, by having AI or the like learn sets of data including confirmed diagnoses and a plurality of examinations performed when the diagnoses were confirmed as well as examination results, it is possible to have a diagnosis confirmed on the basis of results of examinations that have so far been performed and to have a next examination determined on the basis of the examinations that have so far been performed, for the purpose of having a diagnosis confirmed.

Further, in any of the embodiments, when the examinations have ended, the order controlling function 153 may output information presenting the doctor with a diagnosis or treatment predicted in the future to the terminal 200 of the doctor and may output information used by the doctor to give advice to the subject to the terminal 200 of the doctor. For example, when it is learned from an examination related to the disease “liver cancer” suspected in the subject that consumption of alcohol should be prohibited, the order controlling function 153 may output, to the terminal 200 of the doctor, a message reading “It is advised to refrain from consuming alcohol” or the like, as the information used by the doctor to give advice to the subject. As a result, by referring to the information, the doctor is able to give advice to the subject.

Further, the constituent elements of the apparatuses and the devices presented in the drawings in the present embodiments are based on functional concepts. Thus, it is not necessary to physically configure the constituent elements as indicated in the drawings. In other words, specific modes of distribution and integration of the apparatuses and the devices are not limited to those illustrated in the drawings. It is acceptable to functionally or physically distribute or integrate all or a part of the apparatuses and the devices in any arbitrary units, depending on various loads and the status of use. For example, although the medical information processing apparatus 100 is provided in the electronic medical record system 600, possible embodiments are not limited to this example, and the medical information processing apparatus 100 may be provided in a different location within the network. For example, although the processing circuitry 150 is provided in the medical information processing apparatus 100, possible embodiments are not limited to this example, and the processing circuitry 150 may be provided in a different location within the network. For example, the processing circuitry 150 may be provided in the terminal 200 of the doctor. Further, all or an arbitrary part of the processing functions performed by the apparatuses and the devices may be realized by a CPU and a program analyzed and executed by the CPU or may be realized as hardware using wired logic.

Further, it is possible to realize the methods described in the present embodiments, by causing a computer such as a personal computer or a workstation to execute a program prepared in advance. It is possible to distribute the program via a network such as the Internet. Further, it is also possible to record the program onto a non-transitory computer-readable recording medium such as a hard disk, a flexible disk (FD), a Compact Disk Read-Only Memory (CD-ROM), a Magneto-Optical (MO) disk, a Digital Versatile Disk (DVD), or the like, so that the program is executed as being read from the recording medium by a computer.

According to at least one aspect of the embodiments described above, it is possible to improve efficiency of the examinations.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A medical information processing apparatus comprising processing circuitry configured:
to receive, with respect to each of a plurality of examinations for diagnosing a disease suspected in a subject, first information indicating whether or not it is necessary to obtain confirmation from a doctor before the examination is performed and second information indicating whether or not it is necessary to obtain consent from the subject or an appropriate representative before the examination is performed;
to set each of the plurality of examinations with the first information and the second information as additional information; and
to control an examination order including the plurality of examinations on a basis of the additional information.

2. The medical information processing apparatus according to claim 1, wherein
when an examination to be performed is indicated by the first information that it is necessary to obtain the confirmation from the doctor before being performed, the processing circuitry outputs information prompting the doctor to confirm before the examination is performed, and
when an examination to be performed is indicated by the second information that it is necessary to obtain the consent from the subject or the appropriate representative before being performed, the processing circuitry outputs information notifying the doctor that it is necessary to obtain the consent from the subject or the appropriate representative before the examination is performed.

3. The medical information processing apparatus according to claim 1, wherein
the processing circuitry receives, with respect to each of the plurality of examinations, third information indicating whether or not it is necessary to perform an image processing process to detect the disease,
the processing circuitry sets each of the plurality of examinations with the third information as the additional information, and
when the third information indicates that it is necessary to perform the image processing process on a medical image acquired in a performed examination, the processing circuitry causes an automatic diagnosing process to be performed by the image processing process using the medical image.

4. The medical information processing apparatus according to claim 1, wherein
the processing circuitry receives fourth information indicating that it is necessary to obtain confirmation from the doctor, either when a candidate for the disease is changed or when one or more unperformed examinations among the plurality of examinations are not to be performed, on a basis of a result of a performed examination,
the processing circuitry sets the examination order with the fourth information as the additional information, and
when the candidate for the disease is changed or when the one or more unperformed examinations are not to be performed, the processing circuitry outputs information prompting the doctor to confirm on a basis of the fourth information.

5. The medical information processing apparatus according to claim 1, wherein
the processing circuitry receives fifth information indicating that it is necessary to obtain confirmation from the doctor when a result of an image processing process to detect the disease is defective,
the processing circuitry sets the plurality of examinations with the fifth information as the additional information, and
when the result of the image processing process is defective, the processing circuitry outputs information prompting the doctor to confirm on the basis of the fifth information.

6. The medical information processing apparatus according to claim 1, wherein the processing circuitry determines a sequential order of the plurality of examinations on a basis of a schedule of apparatuses that perform the examinations and a schedule of examination staff.

7. The medical information processing apparatus according to claim 1, wherein, when it is impossible to perform one or more of the plurality of examinations at a current point in time due to a schedule of apparatuses that perform the examinations and a schedule of examination staff, the processing circuitry either notifies the doctor or determines a sequential order of the plurality of examinations.

8. The medical information processing apparatus according to claim 1, wherein the processing circuitry changes a sequential order of unperformed examinations while prioritizing an examination that will increase a possibility of detecting the disease, on a basis of a result of a performed examination.

9. The medical information processing apparatus according to claim 1, wherein, on a basis of a result of a performed examination, the processing circuitry cancels one or more of unperformed examinations having a lower possibility of detecting the disease.

10. The medical information processing apparatus according to claim 1, wherein, when a result of a performed examination indicates the disease suspected in the subject, the processing circuitry cancels any unperformed examination.

11. The medical information processing apparatus according to claim 1, wherein, when the plurality of examinations include at least one examination for which it is necessary to obtain the consent from the subject or the appropriate representative before being performed, the processing circuitry outputs a consent form used for obtaining the consent from the subject or the appropriate representative before the examination is performed.

12. The medical information processing apparatus according to claim 1, wherein, among the plurality of examinations, the processing circuitry cancels one or more examinations that were performed within a predetermined time period.

13. A medical information processing system comprising:
a medical information processing apparatus and
a terminal configured to access the medical information processing apparatus and to issue an examination order, wherein
the medical information processing apparatus includes processing circuitry configured:
to receive via the terminal, with respect to each of a plurality of examinations for diagnosing a disease suspected in a subject, first information indicating whether or not it is necessary to obtain confirmation from a doctor before the examination is performed and second information indicating whether or not it is necessary to obtain consent from the subject or an appropriate representative before the examination is performed;
to set each of the plurality of examinations with the first information and the second information as additional information; and
to control the examination order including the plurality of examinations on a basis of the additional information.

14. A medical information processing method comprising:
receiving, with respect to each of a plurality of examinations for diagnosing a disease suspected in a subject, first information indicating whether or not it is necessary to obtain confirmation from a doctor before the examination is performed and second information indicating whether or not it is necessary to obtain consent from the subject or an appropriate representative before the examination is performed;
setting each of the plurality of examinations with the first information and the second information as additional information; and
controlling an examination order including the plurality of examinations on a basis of the additional information.
